(19) United States
(12) Patent Application Publication
(10) Pub. No.: US 2026/0130888 A1
(43) Pub. Date: May 14, 2026

(54) NOVEL THERAPEUTIC MOLECULE

(71) Applicants: Baskaran PILLAI, Carson City, NV (US); Dinesh M.G., Carson City, NV (US); Pillai Universal LLC, Carson City, NV (US)

(72) Inventors: Baskaran PILLAI, Pittsburgh, PA (US); Dinesh M.G., Ambattur, Chennai (IN)

(21) Appl. No.: 19/121,099

(22) PCT Filed: Oct. 12, 2023
(86) PCT No.: PCT/IB2023/060275

§ 371 Date: Apr. 14, 2025

(30) Foreign Application Priority Data
Oct. 15, 2022 (IN) 202241058962

(51) Int. Cl. A61K 31/403; C07D 209/88

(52) U.S. Cl. CPC A61K 31/403; C07D 209/88

(57) ABSTRACT

The present invention discloses a compound of structural formula I

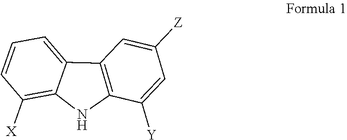

or a pharmaceutically acceptable salt thereof; in which
X is selected from group comprising of unsubstituted or substituted
   1. linear aliphatic alkyl carboxylic acids,
   2. branched aliphatic alkyl carboxylic acids,
   3. linear aliphatic Alkenyl/Alkynyl carboxylic acids,
   4. branched aliphatic Alkenyl/Alkynyl carboxylic acids,
   5. aromatic carboxylic acid and
   6. heteroaryl carboxylic acid;
Y is selected from group comprising of unsubstituted or substituted
   1. linear aliphatic alkoxy group,
   2. branched aliphatic alkoxy group,
   3. linear aliphatic alkenyl/alkynyl oxy group,
   4. branched aliphatic alkenyl/alkynyl oxy group,
   5. aryloxy group and
   6. heteroaryloxy group;
Z is selected from group comprising of unsubstituted or substituted
   1. linear aliphatic alkyl hydroxyl group,
   2. branched aliphatic alkyl hydroxyl group,
   3. linear aliphatic Alkenyl/Alkynyl hydroxyl group,
   4. branched aliphatic Alkenyl/Alkynyl hydroxyl group,
   5. aromatic hydroxyl group and
   6. heteroaryl hydroxyl group.

### FIELD OF THE INVENTION

The present invention generally relates to pharmaceuticals and specifically relates to novel therapeutic drugs. More particularly the present invention relates to novel therapeutic compounds for prevention, treatment and management of Type 2 diabetes mellitus and its related complications.

### BACKGROUND OF THE INVENTION

The global predominance of type 2 diabetes is increasing at an incredible rate. It has been anticipated that by the end of this decade, the number of people having type 2 diabetes will grow to more than 320 million [1]. A range of pharmacological agents is used to improve glucose homeostasis via various modes of action. Biguanides (e.g., metformin) promote glucose utilization and reduce liver glucose production, Sulfonylureas induce insulin secretion, and alpha-glucosidase inhibitors (e.g., acarbose) slow down carbohydrate absorption from the gut, and thiazolidinediones augment cellular level insulin action on glucose metabolism [2]. Insulin replacement therapy is also necessary when insulin production decline in the patients through deprived glycemic control [3]. In current years, treatment policy has to pay attention to the improvement of novel therapeutic options to replace insulin therapy. In Type 2 diabetes, the decreased capability of insulin to stimulate glucose disposal, and the reduced glucose uptake into a muscle or adipose tissues in reaction to insulin, results in a condition referred to as insulin resistance [4]. Despite the molecular basis of Type 2 diabetes is poorly understood, it is well known that insulin signaling, including activation of IR tyrosine kinase activity, is impaired in most patients suffers from Type 2 diabetes [5]. Diverse small compounds like demethylasterriquinone-B1 and TLK19780 have diagnosed as the potent insulin mimetics, but they have poor bioavailability and low receptor specificity [6][7]. As a result, the hunt for new orally dynamic insulin mimetics with stringent receptor selectivity is rather warranted.

### REFERENCES

1. Rao Y K, Lee M, Chen K, Lee Y, Wu W, Tzeng Y. Insulin-Mimetic Action of Rhoifolin and Cosmosiin Isolated from Citrus grandis (L.) Osbeck Leaves: Enhanced Adiponectin Secretion and Insulin Receptor Phosphorylation in 3T3-L1 Cells. 2011; 2011.
2. He K, Chan C B, Liu X, et al. Identification of a molecular activator for insulin receptor with potent anti-diabetic effects. J Biol Chem 2011; 286:37379-37388.
3. Jung D, Ha H, Zheng X, Chang Y, Williams D R. Novel use of fluorescent glucose analogues to identify a new class of triazine-based insulin mimetics possessing useful secondary effects w. 2011:346-358.
4. Kim E Y, Anderson M, Dryer S E. Insulin increases surface expression of TRPC6 channels in podocytes: role of NADPH oxidases and reactive oxygen species. AJP Ren Physiol 2012; 302: F298-F307.
5. Jung S H, Ha Y J, Shim E K, et al. Insulin-mimetic and insulin-sensitizing activities of a pentacyclic triterpenoid insulin receptor activator. Biochem J 2007; 403:243-250.
6. Qiang G, Xue S, Yang J J, et al. Identification of a small molecular insulin receptor agonist with potent antidiabetes activity. Diabetes 2014; 63:1394-1409.
7. Qiang G, Xue S, Yang J J, et al. Identification of a Small Molecular Insulin Receptor Agonist With Potent Antidiabetes Activity. 2014; 63:1394-1409.

### OBJECT OF THE INVENTION

The main object of the present invention relates to novel small molecule for use in for prevention, treatment and management of Type 2 diabetes mellitus and its related complications.

Another object of the present invention is to synthesize novel small molecule for use in for prevention, treatment and management of Type 2 diabetes mellitus and its related complications

Yet another object of the present invention is to synthesize Small-molecule drugs that act by producing insulin-dependent activation of the IR tyrosine kinase domains and are potentially attractive for the treatment of type 2 diabetes.

Yet another object of the present invention is to synthesize Small-molecule drugs with insulin-dependent activity in the control of hyperglycemia by modulation of their effects as insulin levels change in response to physiological stimuli.

Yet another object of the present invention is to synthesize Small-molecule drugs that increases IR autophosphorylation in the presence of insulin and also enhances downstream signaling events, including phosphorylation of IRS-1 and GLUT4 translocation.

Yet another object of the present invention is to synthesize Small-molecule drugs that significantly lowers blood glucose levels in two animal models of type 2 diabetes. Further object of the present invention is utilize the synthesized Small-molecule drugs for the subjects suffering from Type 2 diabetes mellitus and its related complications.

### BRIEF DESCRIPTION OF DRAWINGS

The foregoing and following information as well as other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

FIG. 1 depicts the ¹³C NMR Spectrum of compound I of the present invention

FIG. 2 depicts the ¹H NMR Spectrum of compound I of the present invention

FIG. 3 depicts the IR Spectrum of compound I of the present invention

FIG. 4 depicts the effect of compound I on (a) body weight, (b) Plasma glucose, (c) Total cholesterol and (d) triglyceride levels of HFD+STZ type II diabetic mice

FIG. 5 depicts the Effect of compound I on (a) body weight, (b) Plasma glucose, (c) Total cholesterol and (d) triglyceride levels of db/db diabetic mice

FIG. 6 depicts the level of gene expression in different groups of mice in both Liver and skeletal muscle in the C57BL/6J-db/dbmice

FIG. 7 depicts the Insulin mimicking effect of compound I in Liver and skeletal muscle of HFD+STZ type II diabetic mice
a. Insulin Mimetic Effect
b. Inflammatory mediators

FIG. 8 depicts the effect of compound I on Liver insulin resistance in the C57BL/6J-db/dbmice

FIG. 9 depicts the Insulin mimicking effect of compound I in skeletal muscle and Liver of db/db mice

### SUMMARY OF THE INVENTION

The present invention discloses a compound of structural formula I:

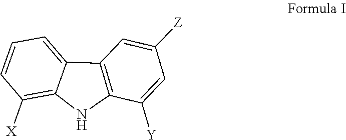

or a pharmaceutically acceptable salt thereof; in which
X is selected from a group comprising of
   1. unsubstituted or substituted linear aliphatic alkyl carboxylic acids,
   2. unsubstituted or substituted branched aliphatic alkyl carboxylic acids,
   3. unsubstituted or substituted linear aliphatic Alkenyl/Alkynyl carboxylic acids,
   4. unsubstituted or substituted branched aliphatic Alkenyl/Alkynyl carboxylic acids,
   5. unsubstituted or substituted aromatic carboxylic acid and
   6. unsubstituted or substituted heteroaryl carboxylic acid;
Y is selected from a group comprising of
   1. unsubstituted or substituted linear aliphatic alkoxy group,
   2. unsubstituted or substituted branched aliphatic alkoxy group,
   3. unsubstituted or substituted linear aliphatic alkenyl/alkynyl oxy group,
   4. unsubstituted or substituted branched aliphatic alkenyl/alkynyl oxy group,
   5. unsubstituted or substituted aryloxy group and
   6. unsubstituted or substituted heteroaryloxy group;
Z is selected from a group comprising of
   1. unsubstituted or substituted linear aliphatic alkyl hydroxyl group,
   2. unsubstituted or substituted branched aliphatic alkyl hydroxyl group,
   3. unsubstituted or substituted linear aliphatic Alkenyl/Alkynyl hydroxyl group,
   4. unsubstituted or substituted branched aliphatic Alkenyl/Alkynyl hydroxyl group,
   5. unsubstituted or substituted aromatic hydroxyl group and
   6. unsubstituted or substituted heteroaryl hydroxyl group;

### DETAILED DESCRIPTION OF THE INVENTION

### Definitions

Alkyl” means saturated carbon chains which may be linear or branched or combinations thereof, unless the carbon chain is defined otherwise. Other groups having the prefix “alk”, such as alkoxy and alkanoyl, also may be linear or branched, or combinations thereof, unless the carbon chain is defined otherwise. Examples of alkyl groups include methyl, ethyl, propyl, isopropyl, butyl, sec- and tert-butyl, pentyl, hexyl, heptyl, octyl, nonyl, and the like. In one embodiment of the present invention, alkyl is methyl.

Alkenyl” means carbon chains which contain at least one carbon-carbon double bond, and which may be linear or branched, or combinations thereof, unless otherwise defined. Examples of alkenyl include vinyl, allyl, isopropenyl, pentenyl, hexenyl, heptenyl, 1-propenyl, 2-butenyl, 2-methyl-2-butenyl, and the like. In one embodiment of the present invention, alkenyl is 2-methyl-1-propenyl.

“Alkynyl” means carbon chains which contain at least one carbon-carbon triple bond, and which may be linear or branched, or combinations thereof, unless otherwise defined. Examples of alkynyl include ethynyl, propargyl, 3-methyl-1-pentynyl, 2-heptynyl and the like. In one embodiment, alkynyl is —C2alkyne-CH3.

“Aryl” means a monocyclic, bicyclic or tricyclic carbocyclic aromatic ring or ring system containing 5-14 carbon atoms, wherein at least one of the rings is aromatic. Examples of aryl include phenyl and naphthyl. In one embodiment of the present invention, aryl is phenyl.

Heteroaryl” means monocyclic, bicyclic or tricyclic ring or ring system containing 5-14 carbon atoms and containing at least one ring heteroatom selected from N, NH, S (including SO and SO2) and O, wherein at least one of the heteroatom containing rings is aromatic. Examples of heteroaryl include pyrrolyl, indole, isoxazolyl, isothiazolyl, pyrazolyl, pyridyl, oxazolyl, oxadiazolyl, thiadiazolyl, thiazolyl, imidazolyl, triazolyl, tetrazolyl, furanyl, triazinyl, thienyl, pyrimidyl, pyridazinyl, pyrazinyl, benzisoxazolyl, benzoxazolyl, benzothiazolyl, benzimidazolyl, benzofuranyl, benzopyrazole (or indazole), benzothiophenyl (including S-oxide and dioxide), furo (2,3-b)pyridyl, quinolyl, indolyl, isoquinolyl, quinazolinyl, dibenzofuranyl, and the like. In one embodiment of the present invention, heteroaryl is selected from: pyridine, pyrazole, thiazole, thiophene, pyrrole, triazole, indazole and indole. In another embodiment of the present invention, heteroaryl is selected from pyrazole, thiazole, thiophene, pyrrole, triazole, indazole and indole. In another embodiment of the present invention, heteroaryl is pyridine.

In choosing compounds of the present invention, one of ordinary skill in the art will recognize that the various substituents, are to be chosen in conformity with well-known principles of chemical structure connectivity and stability.

The term “substituted” shall be deemed to include multiple degrees of substitution by a named substitutent. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

The phrase “pharmaceutically acceptable” is employed herein to refer to those compounds, materials, compositions, salts and/or dosage forms which are, using sound medical judgment, and following all applicable government regulations, safe and suitable for administration to a human being or an animal.

It will be understood that, as used herein, references to the compounds of the present invention are meant to also include the pharmaceutically acceptable salts, and also salts that are not pharmaceutically acceptable when they are used as precursors to the free compounds or their pharmaceutically acceptable salts or in other synthetic manipulations.

The compounds of the present invention may be administered in the form of a pharmaceutically acceptable salt. The term “pharmaceutically acceptable salt” refers to salts prepared from pharmaceutically acceptable non-toxic bases or acids including inorganic or organic bases and inorganic or organic acids. Salts of basic compounds encompassed within the term “pharmaceutically acceptable salt” refer to non-toxic salts of the compounds of this invention which are generally prepared by reacting the free base with a suitable organic or inorganic acid. Representative salts of basic compounds of the present invention include, but are not limited to, the following: acetate, benzenesulfonate, benzoate, bicarbonate, bisulfate, bitartrate, borate, bromide, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, oxalate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate. Furthermore, where the compounds of the invention carry an acidic moiety, suitable pharmaceutically acceptable salts thereof include, but are not limited to, salts derived from inorganic bases including aluminum, ammonium, calcium, copper, ferric, ferrous, lithium, magnesium, manganic, mangamous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, cyclic amines, and basic ion-exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

Also, in the case of a carboxylic acid (—COOH) or alcohol group being present in the compounds of the present invention, pharmaceutically acceptable esters of carboxylic acid derivatives, such as methyl, ethyl, or pivaloyloxymethyl, or acyl derivatives of alcohols, such as O-acetyl, O-pivaloyl, O-benzoyl, and O-aminoacyl, can be employed. Included are those esters and acyl groups known in the art for modifying the solubility or hydrolysis characteristics for use as sustained-release or prodrug formulations.

The term “therapeutically effective” applied to dose or amount refers to that quantity of a compound or pharmaceutical formulation that is sufficient to result in a desired clinical benefit after administration to a patient in need thereof.

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Generally the present invention discloses novel therapeutic compounds in diabetes treatment that Directly Sensitizes the Insulin Receptor.

In one of the preferred embodiment, the present invention shall disclose a compound of structural formula I:

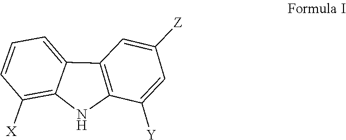

or a pharmaceutically acceptable salt thereof; in which
X is selected from a group comprising of
   1. unsubstituted or substituted linear aliphatic alkyl carboxylic acids,
   2. unsubstituted or substituted branched aliphatic alkyl carboxylic acids,
   3. unsubstituted or substituted linear aliphatic Alkenyl/Alkynyl carboxylic acids,
   4. unsubstituted or substituted branched aliphatic Alkenyl/Alkynyl carboxylic acids,
   5. unsubstituted or substituted aromatic carboxylic acid and
   6. unsubstituted or substituted heteroaryl carboxylic acid;
Y is selected from a group comprising of
   1. unsubstituted or substituted linear aliphatic alkoxy group,
   2. unsubstituted or substituted branched aliphatic alkoxy group,
   3. unsubstituted or substituted linear aliphatic alkenyl/alkynyl oxy group,
   4. unsubstituted or substituted branched aliphatic alkenyl/alkynyl oxy group,
   5. unsubstituted or substituted aryloxy group and
   6. unsubstituted or substituted heteroaryloxy group;
Z is selected from a group comprising of
   1. unsubstituted or substituted linear aliphatic alkyl hydroxyl group,
   2. unsubstituted or substituted branched aliphatic alkyl hydroxyl group,
   3. unsubstituted or substituted linear aliphatic Alkenyl/Alkynyl hydroxyl group,
   4. unsubstituted or substituted branched aliphatic Alkenyl/Alkynyl hydroxyl group,
   5. unsubstituted or substituted aromatic hydroxyl group and
   6. unsubstituted or substituted heteroaryl hydroxyl group;

As per the invention, in the compound of Formula I, the X is unsubstituted branched aliphatic Alkenyl carboxylic acid with at least one carbon atom or a pharmaceutically acceptable salt thereof.

In accordance with the invention, in the compound of Formula I, the X is 1 methyl prop-1-enoic acid.

According to the invention in the compound of Formula I, the Y is unsubstituted linear aliphatic alkoxy group with at least one carbon atom or a pharmaceutically acceptable salt thereof.

As per the invention, in the compound of Formula I, the Y is Methoxy group.

In accordance with the invention, in the compound of Formula I, the Z is unsubstituted linear aliphatic alkyl hydroxyl group with at least one carbon atom or a pharmaceutically acceptable salt thereof.

According to the invention, in the compound of Formula I, the Z is hydroxyl methyl group.

As per the invention, the compound of Formula I, comprises of structure of compound I

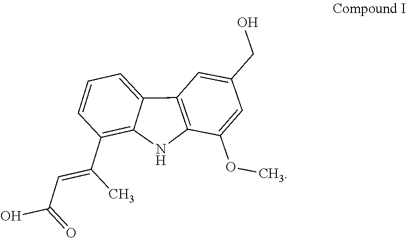 [SMILES: COc1cc(CO)cc2c1[nH]c1c(/C(C)=C/C(=O)O)cccc12]

In another preferred embodiment, the present invention shall disclose a pharmaceutical composition comprising a therapeutically effective amount of compound of Formula I, or a therapeutically effective amount of pharmaceutically acceptable salt thereof, and a pharmaceutically acceptable carrier.

In further preferred embodiment, the present invention shall disclose a pharmaceutical composition comprising a therapeutically effective amount of compound I, and a pharmaceutically acceptable carrier.

In one embodiment the compound I can be prepared by the reaction scheme provided below.

### Stage-1:

### Brief Manufacturing Process:

4-amino-3-methoxybenzoic acid on esterification with Methanol, Thionyl chloride, followed by reaction with and Bis(pyridine)iodonium tetrafluoroborate to affords methyl 4-amino-3-iodo-5-methoxybenzoate.

### Route of Synthesis:

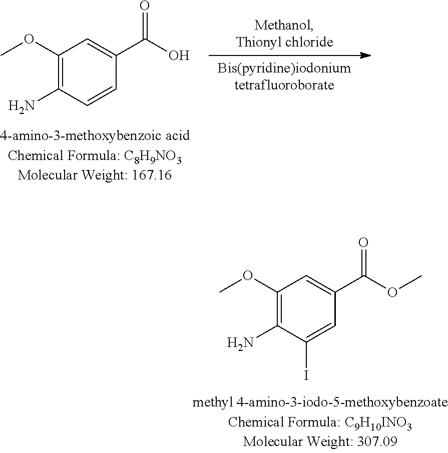

### Stage-2:

### Brief Manufacturing Process:

methyl 4-amino-3-iodo-5-methoxybenzoate on N-acetylation with acetic anhydride in presence of sulfuric acid and Dichloromethane to affords methyl 4-acetamido-3-iodo-5-methoxybenzoate.

### Route of Synthesis:

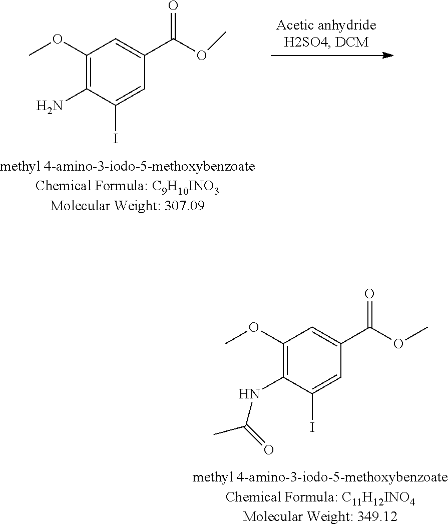

### Stage-3:

### Brief Manufacturing Process:

methyl 4-acetamido-3-iodo-5-methoxybenzoate reacts with phenyl boronic acid in presence of palladium diacetate and 2-Dicyclohexylphosphino-2′,6′-dimethoxybiphenyl to affords methyl 6-acetamido-5-methoxy-[1,1′-biphenyl]-3-carboxylate.

### Route of Synthesis:

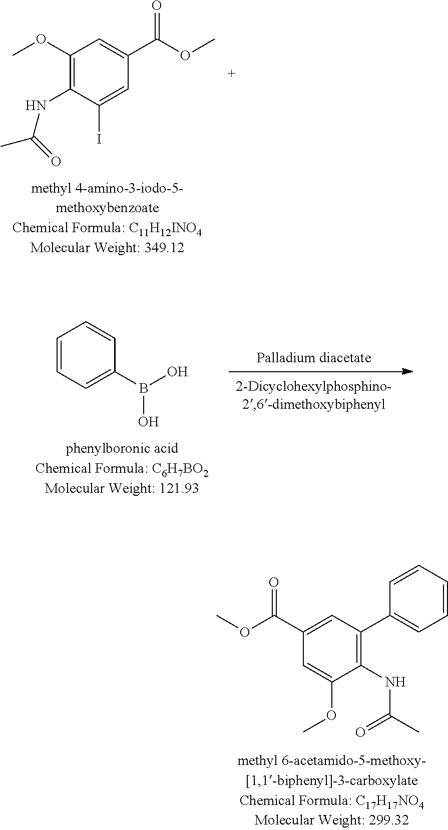 [SMILES: COC(=O)c1cc(I)c(NC(C)=O)c(OC)c1 ; OB(O)c1ccccc1 ; COC(=O)c1cc(OC)c(NC(C)=O)c(-c2ccccc2)c1]

### Stage-4:

### Brief Manufacturing Process:

methyl 6-acetamido-5-methoxy-[1,1′-biphenyl]-3-carboxylate undergoes cyclization in presence of Cupric acetate, Oxygen and palladium acetate to affords methyl 1-methoxy-9H-carbazole-3-carboxylate.

### Route of Synthesis:

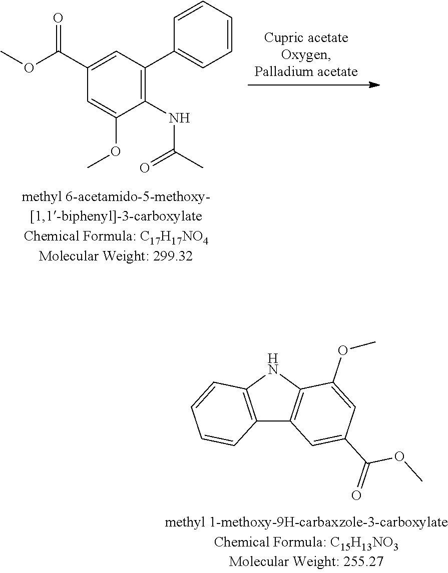 [SMILES: COC(=O)c1cc(OC)c(NC(C)=O)c(-c2ccccc2)c1 ; COC(=O)c1cc(OC)c2[nH]c3ccccc3c2c1]

### Stage-5:

### Brief Manufacturing Process:

methyl 1-methoxy-9H-carbazole-3-carboxylate reacts with acetyl chloride to affords methyl 8-acetyl-1-methoxy-9H-carbazole-3-carboxylate

### Route of Synthesis:

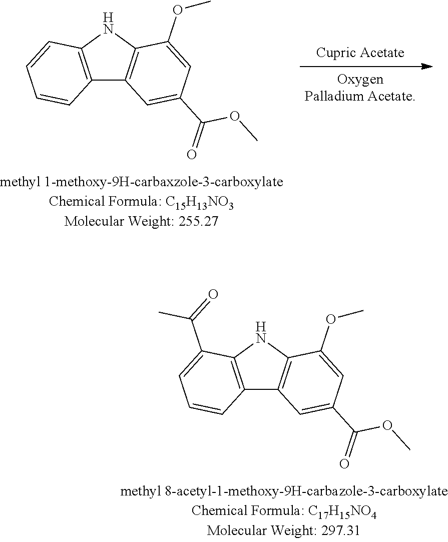

### Stage-6:

### Brief Manufacturing Process:

methyl 8-acetyl-1-methoxy-9H-carbazole-3-carboxylate reacts with Ethyl triphenyl phosphoranyliden acetate in presence of sodium hydroxide and Hydrochloric acid to affords (E)-3-(8-methoxy-6-(methoxycarbonyl)-9H-carbazol-1-yl) but-2-enoic acid, followed by carboxylic acid protection with 2-yl-methyl-1,3-dithian and reduction with LiAlH₄ and finally deprotection of carboxylic acid with Sodium periodate/Potassium Carbonate to form (2E)-3-[6-(hydroxymethyl)-8-methoxy-9H carbazol-1-yl]but-2-enoic acid (Compound 1).

### Route of Synthesis:

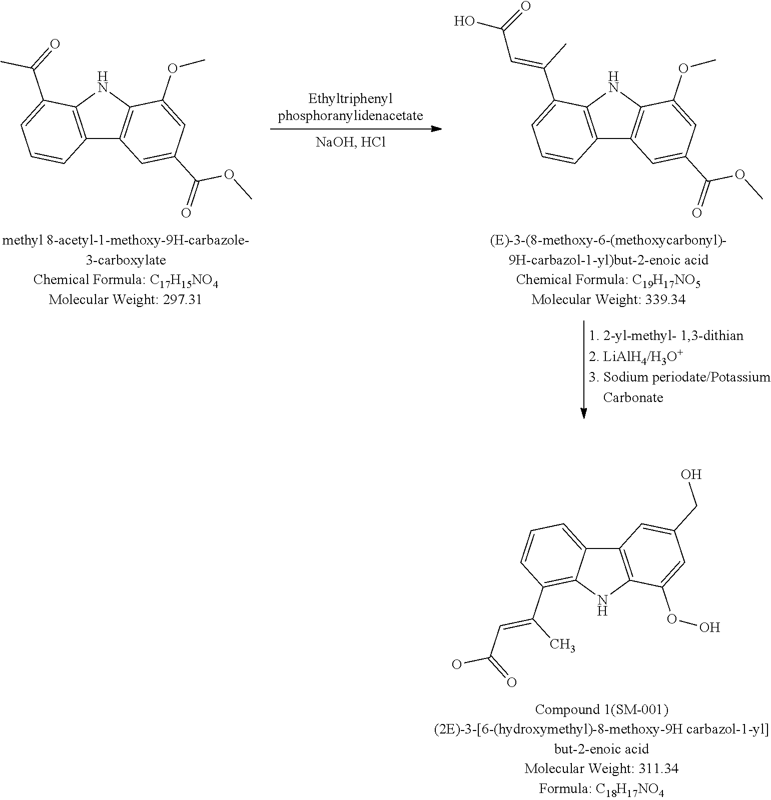 [SMILES: COC(=O)c1cc(OC)c2[nH]c3c(C(C)=O)cccc3c2c1 ; COC(=O)c1cc(OC)c2[nH]c3c(/C(C)=C/C(=O)O)cccc3c2c1 ; C/C(=C\C(=O)O)c1cccc2c1[nH]c1c(OO)cc(CO)cc12]

The synthesized compound I is next subject to molecular characterization to ascertain the structure of the compounds.

### Molecules Characterization:

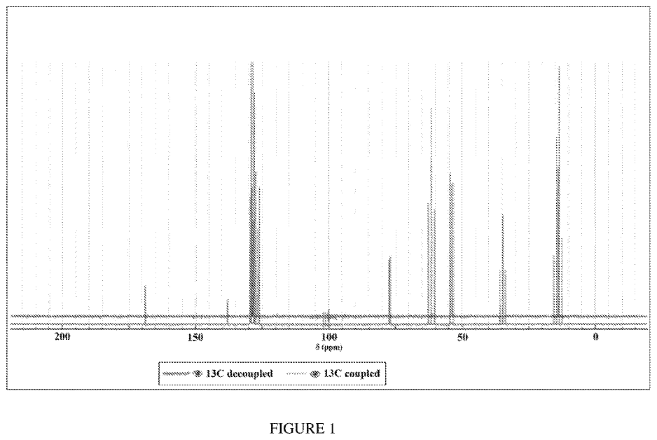

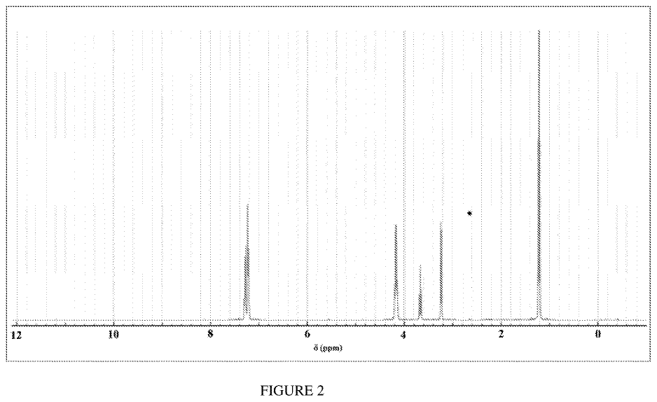

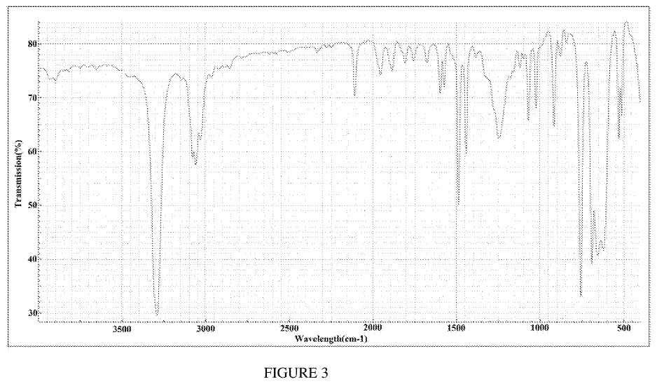

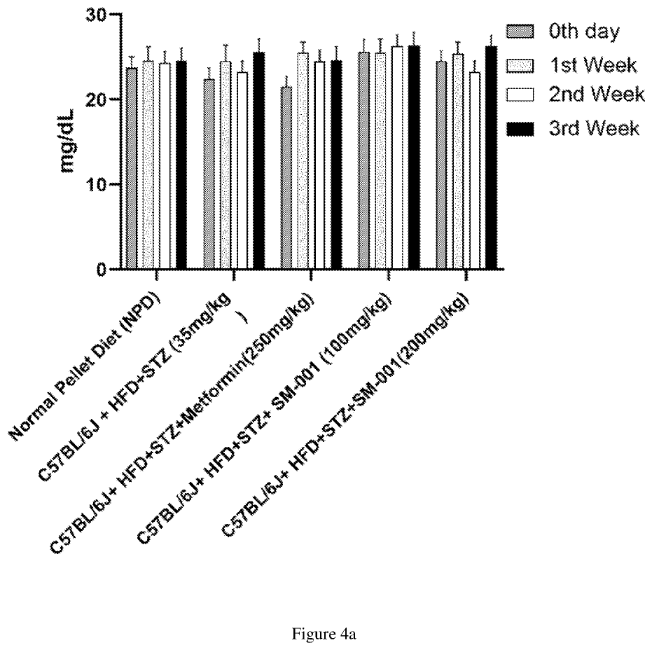

The purity of synthesized Compound I was verified by the melting point, Thin Layer Chromatography, HPLC, IR, Mass Spectroscopy and NMR analysis. From FIG. 1-3, the structure of the synthesized compound I is elucidated as follows.

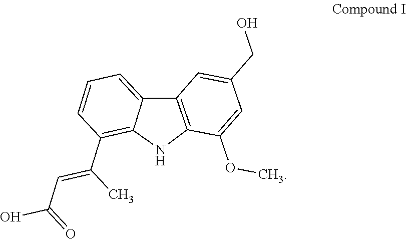 [SMILES: COc1cc(CO)cc2c1[nH]c1c(/C(C)=C/C(=O)O)cccc12]

In some embodiments, the compound I may encompass both the cis- and trans-isomers. In some embodiments, the compound I may be a mixture of cis- and trans-isomers. In some embodiments, the compound I may be cis-isomer. In some embodiments, the compound I may be trans-isomer.

In some embodiments, the compound I may encompass either R or S stereoisomers and a mixture of stereoisomers. In some embodiments, the compound I may encompass both racemic isomers and enantiomeric isomers

The compound I of the present inventions can be used to perform or provide any of the biological functions, described herein.

### Pharmaceutical Compositions

The present disclosure also includes pharmaceutical compositions comprising a therapeutically effective amount of compound I disclosed herein. In some embodiments, pharmaceutical compositions comprise a therapeutically effective amount of compound I or pharmaceutically acceptable salts thereof.

In various aspects, the amount of compound I, or a pharmaceutically acceptable salt thereof, can be administered at about 0.001 mg/kg to about 100 mg/kg body weight (e.g., about 0.01 mg/kg to about 10 mg/kg or about 0.1 mg/kg to about 5 mg/kg).

The concentration of a disclosed compound in a pharmaceutically acceptable mixture will vary depending on several factors, including the dosage of the compound to be administered, the pharmacokinetic characteristics of the compound(s) employed, and the route of administration. The agent may be administered in a single dose or in repeat doses. The dosage regimen utilizing the compounds of the present invention is selected in accordance with a variety of factors including type, species, age, weight, sex and medical condition of the patient; the severity of the condition to be treated; the route of administration; the renal and hepatic function of the patient; and the particular compound or salt thereof employed. Treatments may be once administered daily or more frequently depending upon a number of factors, including the overall health of a patient, and the formulation and route of administration of the selected compound(s).

The compounds or pharmaceutical compositions of the present disclosure may be manufactured and/or administered in single or multiple unit dose forms.

In some embodiments, the compound I of the present disclosure are administered to a patient with a Type 2 diabetes mellitus and its related complications.

In certain embodiments, the compounds, and compositions described herein are administered in combination with one or more of antidiabetic drug. Compound I of the present invention may be used in combination with other drugs that may also be useful in the treatment or amelioration of the diseases or conditions for which compounds of the present invention are useful. Such other drugs may be administered, by a route and in an amount commonly used therefor, contemporaneously or sequentially with a compound of the present invention. In the treatment of patients who have Type 2 diabetes, insulin resistance, obesity, metabolic syndrome, and co-morbidities that accompany these diseases, more than one drug is commonly administered. The compounds of this invention may generally be administered to a patient who is already taking one or more other drugs for these conditions. Often the compounds will be administered to a patient who is already being treated with one or more antidiabetic compound, such as metformin, sulfonylureas, and/or PPARγ agonists, when the patient's glycemic levels are not adequately responding to treatment.

When compound I of the present invention is used contemporaneously with one or more other drugs, a pharmaceutical composition in unit dosage form containing such other drugs and the compound of the present invention is preferred. However, the combination therapy also includes therapies in which the compound I of the present invention and one or more other drugs are administered on different overlapping schedules. It is also contemplated that when used in combination with one or more other active ingredients, the compound of the present invention and the other active ingredients may be used in lower doses than when each is used singly. Accordingly, the pharmaceutical compositions of the present invention include those that contain one or more other active ingredients, in addition to a compound of the present invention.

Examples of other active ingredients that may be administered separately or in the same pharmaceutical composition in combination with compound I described herein include, but are not limited to: other dipeptidyl peptidase-IV (DPP-4) inhibitors, insulin sensitizers, insulin or insulin analogs, leptin and leptin derivatives and agonists, amylin and amylin analogs, sulfonylurea and non-sulfonylurea insulin secretagogues, α-glucosidase inhibitors, glucagon receptor antagonists, incretin mimetics, LDL cholesterol lowering agents, HDL-raising drugs, antiobesity compounds, anti-inflammatory drugs, antihypertensive agents, glucokinase activators, inhibitors of 11β-hydroxysteroid dehydrogenase type 1, CETP inhibitors, inhibitors of fructose 1,6-bisphosphatase, inhibitors of acetyl CoA carboxylase-1 or 2, AMP-activated Protein Kinase (AMPK) activators, other agonists of the G-protein-coupled receptors, SSTR3 antagonists, neuromedin U receptor agonists, SCD inhibitors, GPR-105 antagonists, SGLT inhibitors, inhibitors of acyl coenzyme A, inhibitors of fatty acid synthase, inhibitors of acyl coenzyme A, agonists of the TGR5 receptor, ileal bile acid transporter inhibitors, PACAP, PACAP mimetics, and PACAP receptor 3 agonists, PPAR agonists, protein tyrosine phosphatase-1B (PTP-1B) inhibitors, IL-1b antibodies, bromocriptine mesylate and rapid-release formulations thereof, GPR 120 agonists, anti-diabetic agents, anti-dyslipidemic agents, anti-hypertensive agents, anti-obesity agents and anorectic agents.

The present invention also provides a method for the treatment or prevention of Type 2 diabetes mellitus and its related complications, which method comprises administration to a patient in need of such treatment or at risk of developing a Type 2 diabetes mellitus and its related complications, of therapeutically effective amount Compound I of the present invention and an amount of one or more active ingredients, such that together they give effective relief.

In a further aspect of the present invention, there is provided a pharmaceutical composition comprising a Compound I of the present invention together with at least one pharmaceutically acceptable carrier or excipient.

Thus, according to a further aspect of the present invention there is provided the use of Compound I of the present invention for the manufacture of a medicament for the treatment or prevention of Type 2 diabetes mellitus and its related complications. In a further or alternative aspect of the present invention, there is therefore provided a product comprising a Compound I of the present invention and one or more active ingredients as a combined preparation for simultaneous, separate or sequential use in the treatment or prevention of a Type 2 diabetes mellitus and its related complications.

It will be appreciated that for the treatment or prevention of diabetes, and its related complications, a compound of the present invention may be used in conjunction with another pharmaceutical agent effective to treat that disorder.

The present invention also provides a method for the treatment or prevention of diabetes, and its related complications, which method comprises administration to a patient in need of such treatment an amount of a compound I of the present invention and an amount of another pharmaceutical agent effective to threat that disorder, such that together they give effective relief.

The present invention also provides a method for the treatment or prevention of diabetes, and its related complications, which method comprises administration to a patient in need of such treatment an amount of a compound I of the present invention and an amount of another pharmaceutical agent useful in treating that particular condition, such that together they give effective relief.

### BIOLOGICAL EXAMPLES

### Example 1

Effect of Compound I on body weight, Total cholesterol, Plasma glucose, and triglyceride levels of HFD+STZ type II diabetic mice:

Eight weeks of dietary manipulation with high-fat diet increased the body weight in all the groups when compared to NPD group. Administration of low dose STZ significantly decreased (p<0.05) the body weight in the HFD+STZ diabetic group when compared to their day 0 weight during the experiment. When Compound I of the present invention (100 mg/kg and 200 mg/kg b.wt) administrated to the C57BL/6J mice fed high-fat diets for 14 days, food intake of the induced mice was drastically lower than those of the control mice (p<0.001). Also, body weights significantly reduce in the Compound I-treated mice as compared with the control mice. Administration of Standard Metformin (250 mg/kg b.wt) markedly reduced the rates of glucose, total cholesterol, and triglycerides as compared to the diabetic group. Low and high doses of Compound I showed approximately two fold decrease in the Plasma glucose and triglycerides levels, whereas the total cholesterol levels were reduced by 2.5 fold when compared to the diabetic group. Fasting plasma glucose, insulin, and triglyceride levels markedly diminished in the Compound I-treated mice than the control mice at the 14 days after treatment in a dose-dependent manner FIG. (4 a,b,c,d).

### Example 2

Effect of Compound I on Body Weight Total Cholesterol, Plasma Glucose, and Triglyceride Levels of db/db Diabetic Mice

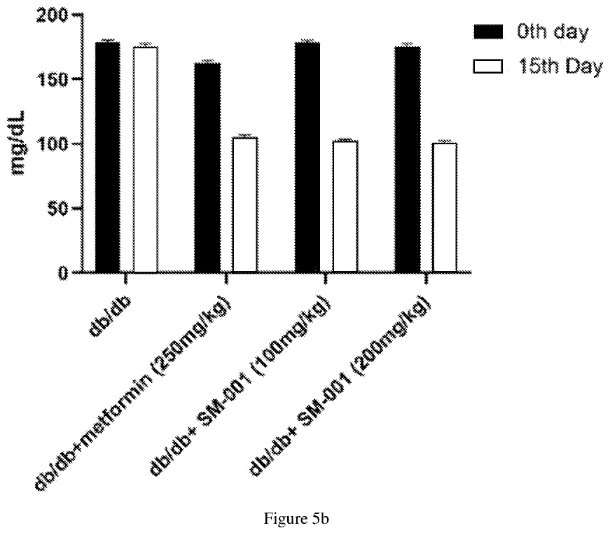

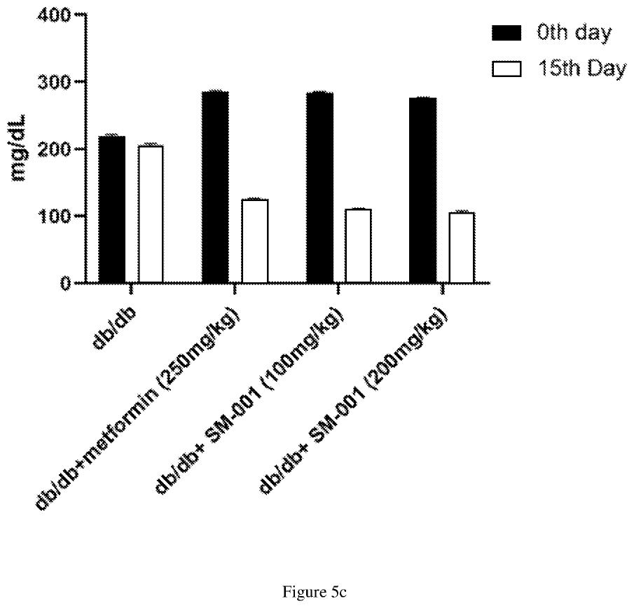

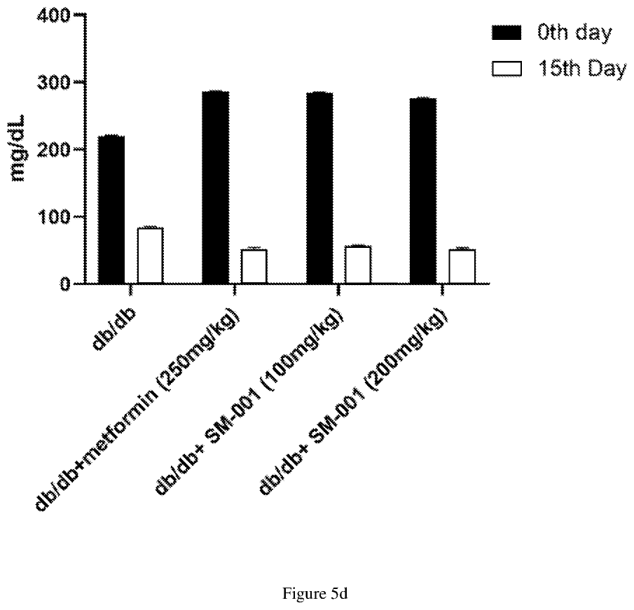

When Compound I (100 mg/kg and 200 mg/kg b.wt) administrated to the db/db mice for 2 weeks, food intake of the drug induced mice was extensively lower than those of the control mice (p>0.05). Also, body weights were also significantly decreased in the-treated mice as compared with the control mice (p>0.05). It is well-known that there is an increase of serum lipid concentration in people with diabetes. As shown in FIG. 5, the db/db mice used in this study had considerably prominent triglycerides and total cholesterol concentrations compared to the control mice (P<0.01, P<0.05). The serum triglyceride and total cholesterol levels in the metformin-treated group were lower than in the model group (P<<0.05). The Compound I treated mice lowered the total cholesterol and triglycerides concentration in a dose-dependent manner, and the high-dose treated group showed a better lipid lowering profile (P<0.05) than other dose groups. Fasting plasma glucose, triglyceride levels, and insulin were markedly lower in the Compound I treated mice with control mice in the 7 days aftertreatment (P>0.001). These data propose that Compound I could capably restore serum glucose, insulin, and lipid abnormalities without significant body weight gain.

### Example 3

### The Effect of Compound I on the Expression of Diabetic-Associated Gene Transcripts in Streptozotocin-Induced Diabetic Rats:

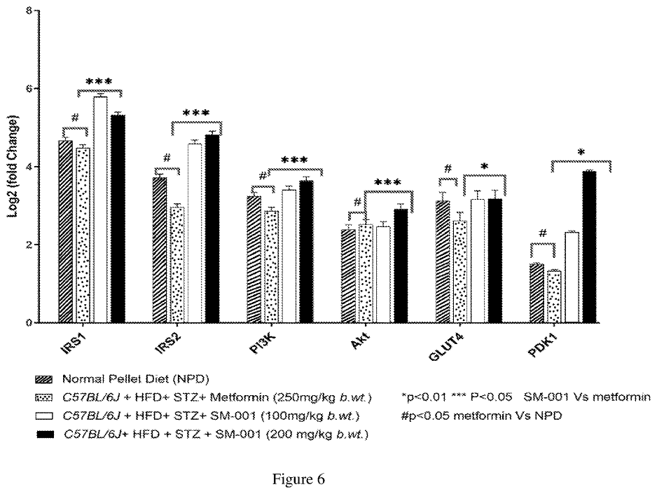

One of the mechanisms which can stimulate or enhance glucose uptake in cells is the elevated translocation of glucose transporter 4 (GLUT 4) from the intracellular site to the plasma membrane. This effect will be mediated through insulin signaling pathway as suggested by gene expression profile. The expression of diabetic-related, gluconeogenic and glycolytic genes, in streptozotocin-induced diabetic mice, were treated with Compound I was determined using semiquantitative RT-PCR. As the internal control, GAPDH the band density of each target gene normalized to the band density of GAPDH of the sample. FIG. 6 showed the level of gene expression in different groups of mice in both Liver and skeletal muscle. Based on Fold change treatment with the STZ-35 mg/kg showed significantly decreased expression of genes involved in insulin signaling pathways such as IRS-1, IRS-2, PI3K, Glut4 and Akt in both liver and skeletal muscles compared to the control. Metformin-treated groups exhibited significantly (p<0.05) increased levels of insulin-signaling gene expression in both skeletal muscle and liver tissue. However, treatment with Compound I at 100 mg/kg enhanced the expression of all the gene transcripts by ˜3.5 fold change in skeletal muscle, and ˜2.5 fold increased expression in Liver tissue respectively. These results suggest that Compound I stimulate the genes involved in insulin signaling pathway which may account for the antihyperglycemic effects. The treatments with all the tested formulation aid in overcoming the insulin resistance activity by restoring the above insulin dependent signaling gene expressions levels to normal.

### Example 4

### Insulin Mimicking Effect of Compound I in Liver and Skeletal Muscle of HFD+STZ Type II Diabetic Mice

In the insulin signaling pathway, protein tyrosine phosphatase 1B (PTP1B) considered playing a vital role in the intracellular signal transduction process and metabolism. There is compelling confirmation that PTP1B, is principally responsible for the dephosphorylation of insulin receptor (IR-β), as a result, leading to the block of insulin signaling transduction pathway. The study investigated whether the Compound I possess insulin mimicking activity on HFD+STZ-induced mice by PCR analysis. As shown in FIG. 7 diabetic induced group exhibited an increased PTP1B activity in liver and skeletal muscle. Whereas treatment with the Compound I (p<0.05) significantly decreased the expression of PTP1B with a concomitant increase in the IR-β levels in a dose-dependent manner, thereby revealing the insulin mimicking property of the Compound I. Treatment with Compound I significantly reduced the expression of inflammatory parameters. (FIG. 7, a & b)

### Example 5

### Effect of Compound I on Liver Insulin Resistance in the C57BL/6J-db/Dbmice

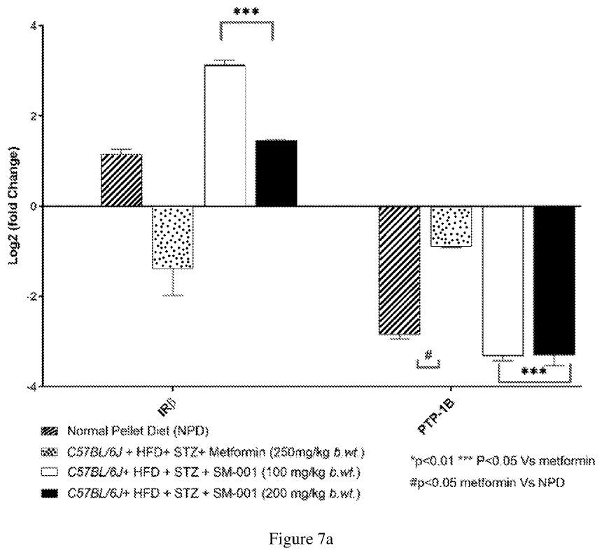

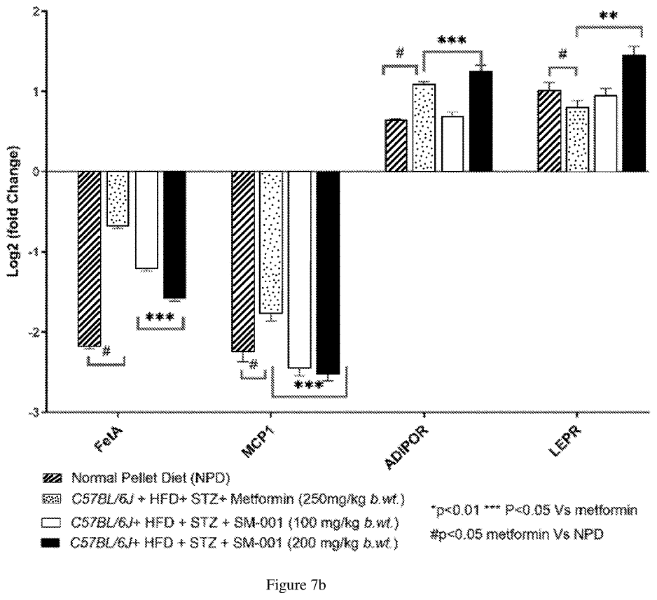

The expression of insulin signaling genes such as IRS-1, IRS-2, PI3K, Glut4, Akt in the livers of the db/db mice reduced noticeably when compared with that of the normal mice. There was a significant enhance within the activation of all the examined genes in the C57BL/6J-db/db treatment group compared to that of normal control group FIG. 8. Supplementation with Compound I notably enhanced the expression of all genes involved in insulin signaling pathway in comparison with that of the C57BL/6J-db/db control group (p<0.05).

### Example 6

Insulin Mimicking Effect of COMPOUND I in Skeletal Muscle and Liver of db/db Mice

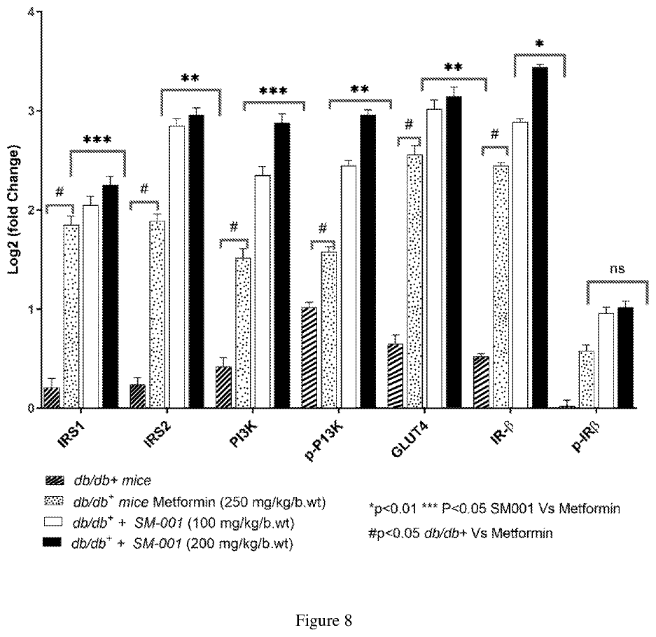

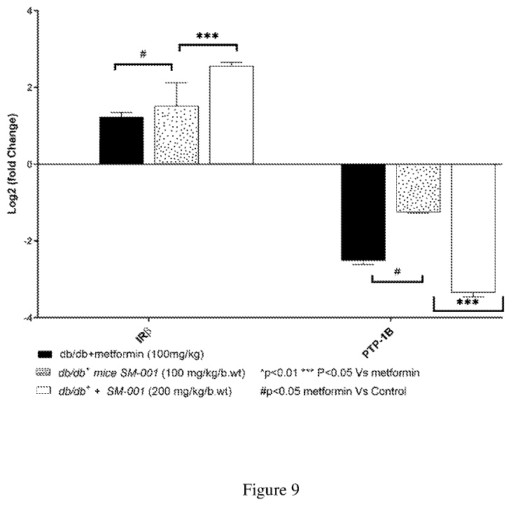

To further utilize the insulin mimetic effect of Compound I, the key proteins in PTP1B and PI3K/Akt signaling pathway were evaluated using Western blot and PCR after treatment with Compound I. Western blotting and PCR analysis have been carried out to verify the expression of PTP1B and IR β in skeletal muscle and liver to establish the inhibition effect of Compound I towards PTP1B. PTP1B in the Compound I treated groups was downregulated substantially with contrast to the control group (p<0.01) (FIG. 9). Additionally, p-IRβ in skeletal muscle and liver significantly increased in Compound I treated group, compared with non-treated control group (p<0.0); this information proposed the mechanism of Compound I that inhibits PTP1B and trigger the insulin receptor signal in vivo.

### Discussion on Biological Examples 1-6

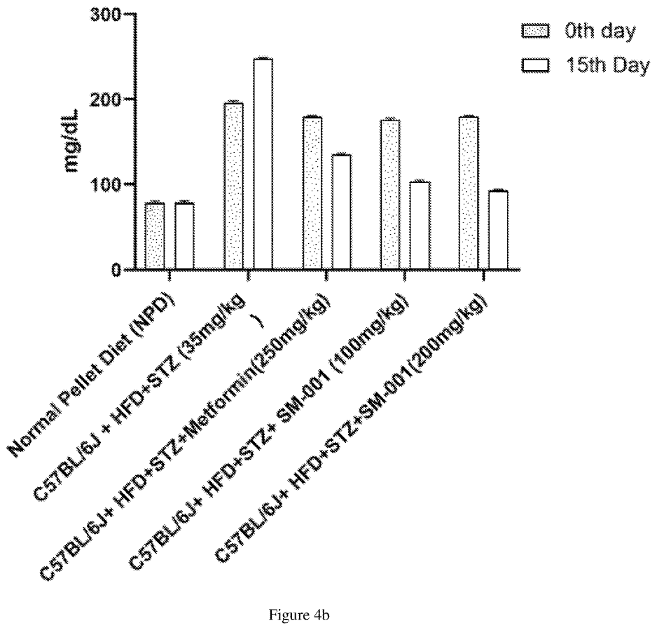

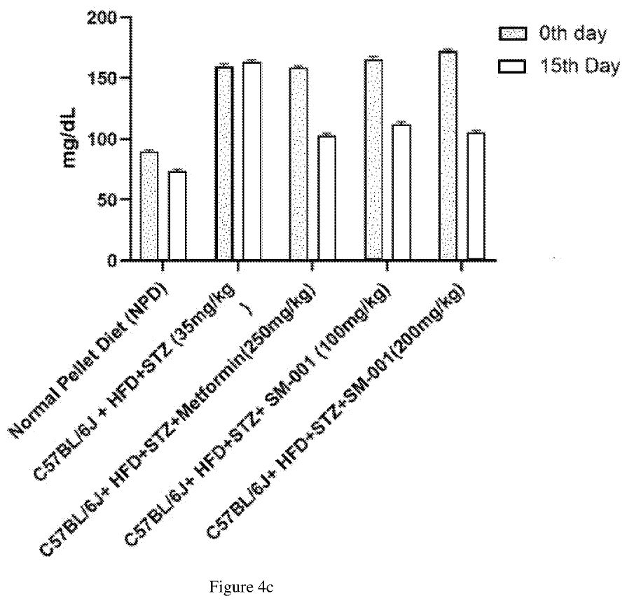

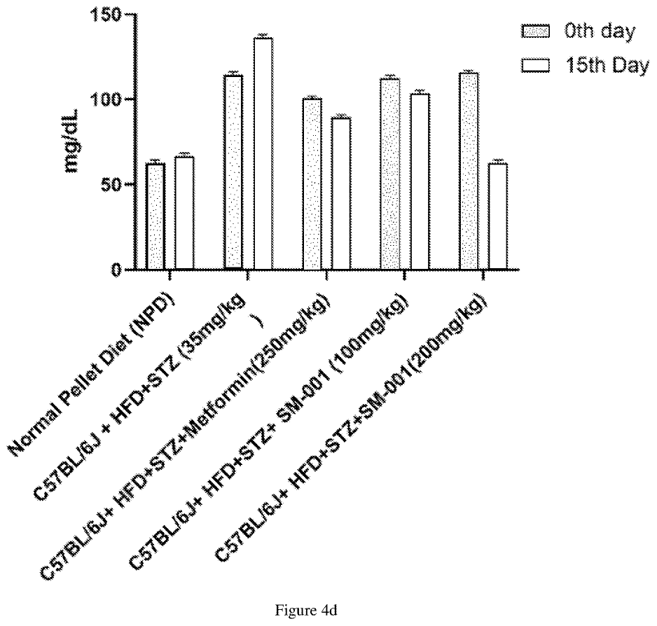

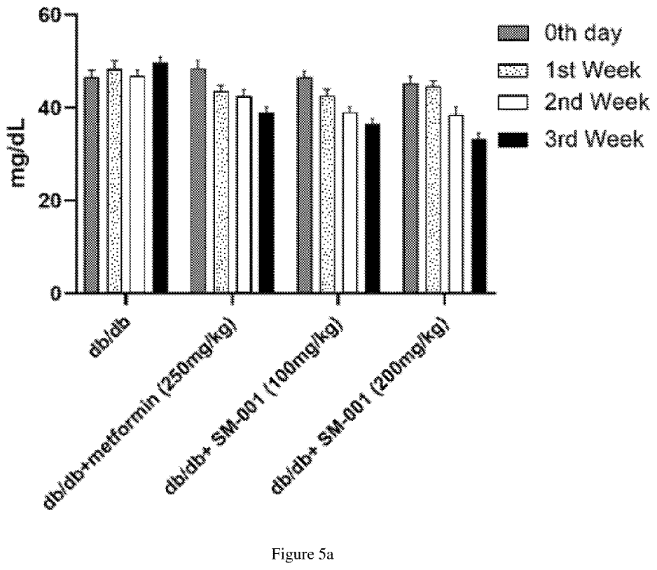

Type 2 diabetes mellitus and its related complications have arisen as serious health problems in modern societies. The cutting edge technology of drug development is to identify small molecule drugs helpful in controlling both diabetes and obesity. In this study, we assess the impact of a small molecule Compound I having insulin mimicking effect by inhibiting PTP-1B and activating IR-β phosphorylation in insulin-sensitive tissues by in vivo animal model. Streptozotocin selectively destroyed the pancreatic insulin secreting b-cells, leaving the less active cell and resulting in a diabetic state (Szkudelski 2001). Intra-peritoneal administration of streptozotocin in the present study efficiently induced diabetes mellitus in mice. The induction of diabetes mellitus in mice confirmed by elevated levels of fasting plasma glucose. Plasma glucose level measured in normal and experimental mice. STZ-treated diabetic mice showed a significant increase in the levels of blood glucose when compared to normal mice. Oral administration of Compound I 100 mg/kg and 200 mg/kg b.wt showed the highly significant effect by reducing the plasma glucose level (FIG. 4, a-d). Furthermore, insulin levels and triglyceride levels were markedly reduced in the Compound I-treated mice than the normal control mice at the 14 days after treatment in a dose-dependent manner (FIG. 4 a-d). When Compound I (100 mg/kg and 200 mg/kg b.wt) administered to the C57BL/6J mice fed high-fat diets for 7 days, food intake of the treated mice was extensively decreased than those of the control mice. Additionally, body weights have also been widely dropped in the Compound I-treated mice as compared with the normal control mice. Insulin levels, Fasting plasma glucose levels, and triglyceride levels were markedly diminished in the Compound I-treated mice compared to control mice on the 7 days after injection.

db/db mouse is a genetic obesity diabetic animal model produced with the aid of the shortage of leptin receptor. The pathogenesis features of T2DM in db/db mice was similar to those in human type 2 diabetes, including hyperglycemia, obesity, and insulin resistance. Current years, db/db mice have been broadly used in animal experiments to set up the T2DM model. We have investigated whether Compound I include IR activators with insulin-mimetic and insulin-sensitizing activity by means of evaluating their effects on IR signaling. Compound I (100 mg/kg and 200 mg/kg b.wt) treatment for 7 days reduced food consumption and body weight in the db/db mice, an inherited obese animal model (FIG. 5, a-d). Treatment additionally reduced, insulin levels, fasting plasma glucose levels, free fatty acids levels and triglyceride level in the db/db mice compared with the normal control. Based on these findings, further studies on Compound I have been carried out in the subsequent experiments to explore their inhibitory activity against PTP1B.

Deficiencies within the IR and its signal transduction pathway have been discovered in insulin-resistant patients, comprising reduced Insulin Receptor- and Insulin Receptor Substrate (IRS)-1-phosphorylation and decreased PI3K activity. Impede insulin signaling lead to hyperglycemia and other various metabolic abnormalities. Therefore, pharmacological agents that augment IRβ tyrosine kinase receptor activity might be helpful in the treatment of Type 2 diabetes, that is considered using irregular insulin secretion due to diminishing β-cell function and insulin resistance in target tissues.

The progression of tyrosine phosphorylation and dephosphorylation is the core mechanism of cell growth and differentiation, and the balance of this process maintained by protein tyrosine phosphatase (PTP1b) and protein tyrosine kinase (PTK). Protein tyrosine phosphatase 1B (PTP1B) is a vital member of the family, a negative regulator in insulin signal transduction and a potential target for treatment of type 2 diabetes mellitus. Moreover, PTP1B could dephosphorylate activated JAK2 and STAT3 and prevented leptin signal transduction. Increased expression of PTP1B influenced the activity of PTKs, which resulted in failing of insulin to bind with IR, induced the insulin resistance and leptin resistance, and caused type 2 diabetes and obesity.

Next, we examine the effect of COMPOUND Ion PTP-1B inhibition and activation of IR-β phosphorylation by PCR and Western blotting studies. In the current study, we used the type 2 diabetic C57BL/6J STZ, HFD+STZ C57BL/6J, db/db mouse model to assess the antihyperglycemic property. FIGS. 6 and 8 demonstrate an increased expression of PTP1B in an induced animal by western blotting and PCR Analysis. As shown in FIGS. 6&8, There is a diminish in the tyrosine phosphorylation levels of the IR β- in the skeletal muscle and liver of diabetic mice, involving the PTP1B function in all the diabetic-treated mice. According to Tagami et al. expression of PTP1B activity up regulated in the skeletal muscle and adipose tissue of Otsuka Long-Evans Tokush-in fat rats. Haj et al. established that the hepatic-specific over expression of PTP1B confirmed insulin resistance no longer merely in the liver but also in other tissues in PTP1B knockout mice. For that reason, PTP1B plays a main function in diabetic mice. When PTP1B gets activated, the insulin signaling transduction pathway blocked, and the level of plasma glucose is disturbed, ensuing in the increased plasma glucose level. As shown in FIG. 6, phosphorylated forms of insulin-signaling molecules, such as p-IRβ, IR, were drastically reduced in the muscles and liver of diabetic group, compared with the normal group. However, oral administration of Compound I significantly augmented the expression level of proteins in a dose-dependent manner. Furthermore, GLUT4 mRNA expression was also extremely expressed in the compound I administered group, compared with that of normal control group (FIG. 4). The increased in the tyrosine phosphorylation expression level of the IR β-subunit in the skeletal muscle and liver of Compound I treated diabetic mice point out the enhancement of insulin sensitivity in the entire animal model tested.

PI3K, as a key molecule in insulin dependent signaling pathway, has been found to play a crucial role in diabetes. In this study, we also examine Compound I enhanced the expression of PI3K in skeletal muscle and liver tissue which may be able to improve insulin transduction through the PI3K/Akt signaling pathway. These findings were by the study in liver tissue of diabetic mice, showing that PI3K diminished in diabetic rats associated with normal rats. Also, the increased p13K led to the enrichment of GLUT4. Investigation of the mechanisms of the Compound I to protect against T2DM inspected by analyzing the expression level of the gene in the PI3K/Akt signaling cascade. Gene expression study indicated that PI3K and GLUT4 expression levels were enhanced significantly with compound I treatments in contrast to diabetic mice. Based on these results were in line with the followed reports. Shih et al. discovered GLUT4 in skeletal muscle were greater indicated groups than diabetic group.

Therefore, we propose that Compound I reduces the expression profile and activities of PTP1B in the insulin-sensitive tissues of the liver and skeletal muscle. Because of the reduction in both the expression and the relative active ratio, most critical to the increase in the expression of tyrosine phosphorylation level of the IR β-subunit and the development of insulin sensitivity. To this point, no PTP1B inhibitor along with Compound I has been found to be efficient in multi-tissues in vivo.

In conclusion, this study showed that Compound I has insulin-mimicking bioactivities and improves glucose tolerance in all the mice models of diabetes with or without obesity condition. Such as male C57BL/6J mice fed normal diets (standard model), mice fed with high-fat diets (an animal model of diabetes with mild obesity), db/db mice (an inherited model of diabetes with severe obesity), STZ-diabetic mice (model of lean insulin-deficient diabetes), respectively. In this report, we have identified a novel small molecule Compound I, which mimics the capabilities of insulin to inhibit PTP-1B and activates Insulin Receptor and its downstream signaling molecules in vitro and in vivo. This study presents for the first time that Compound I mimic the biological actions of insulin by specifically binding to IR to increase its kinase activity and exert their antidiabetic consequences that lower blood glucose in both normal and insulin-resistant mice thus improving glucose uptake in insulin-sensitive tissue by way of IRB phosphorylation. Thus, Compound I is a suitable candidate for the management of diabetes mellitus and the associated metabolic disorders.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope being indicated by the following claims.

We claim:

1. A compound of structural formula I: 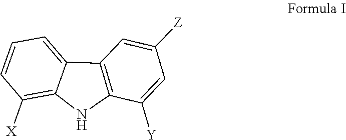
or a pharmaceutically acceptable salt thereof; wherein
X is selected from a group comprising of
1. unsubstituted or substituted linear aliphatic alkyl carboxylic acids,
2. unsubstituted or substituted branched aliphatic alkyl carboxylic acids,
3. unsubstituted or substituted linear aliphatic Alkenyl/Alkynyl carboxylic acids,
4. unsubstituted or substituted branched aliphatic Alkenyl/Alkynyl carboxylic acids,
5. unsubstituted or substituted aromatic carboxylic acid and
6. unsubstituted or substituted heteroaryl carboxylic acid;
Y is selected from a group comprising of
1. unsubstituted or substituted linear aliphatic alkoxy group,
2. unsubstituted or substituted branched aliphatic alkoxy group,
3. unsubstituted or substituted linear aliphatic alkenyl/alkynyl oxy group,
4. unsubstituted or substituted branched aliphatic alkenyl/alkynyl oxy group,
5. unsubstituted or substituted aryloxy group and
6. unsubstituted or substituted heteroaryloxy group;
Z is selected from a group comprising of
1. unsubstituted or substituted linear aliphatic alkyl hydroxyl group,
2. unsubstituted or substituted branched aliphatic alkyl hydroxyl group,
3. unsubstituted or substituted linear aliphatic Alkenyl/Alkynyl hydroxyl group,
4. unsubstituted or substituted branched aliphatic Alkenyl/Alkynyl hydroxyl group,
5. unsubstituted or substituted aromatic hydroxyl group and
6. unsubstituted or substituted heteroaryl hydroxyl group;

2. The compound as claimed in claim 1 wherein the said X is unsubstituted branched aliphatic Alkenyl carboxylic acid with at least one carbon atom or a pharmaceutically acceptable salt thereof.

3. The compound as claimed in claim 1 wherein the said X is 1 methyl prop-1-enoic acid.

4. The compound as claimed in claim 1 wherein the said Y is unsubstituted linear aliphatic alkoxy group with at least one carbon atom or a pharmaceutically acceptable salt thereof.

5. The compound as claimed in claim 1 wherein the said Y is Methoxy group.

6. The compound as claimed in claim 1 wherein the said Z is unsubstituted linear aliphatic alkyl hydroxyl group with at least one carbon atom or a pharmaceutically acceptable salt thereof.

7. The compound as claimed in claim 1 wherein the said Z is hydroxyl methyl group.

8. The compound as claimed in claim 1 comprises of structure of compound I

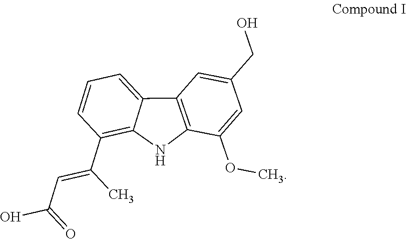 [SMILES: COc1cc(CO)cc2c1[nH]c1c(/C(C)=C/C(=O)O)cccc12]

9. A pharmaceutical composition comprising a therapeutically effective amount of compound of claim 1, or a therapeutically effective amount of pharmaceutically acceptable salt thereof, and a pharmaceutically acceptable carrier.

10. A pharmaceutical composition comprising a therapeutically effective amount of compound of claim 8, and a pharmaceutically acceptable carrier.
